(19) United States
(12) Patent Application Publication
(10) Pub. No.: US 2011/0070630 A1
(43) Pub. Date: Mar. 24, 2011

(54) METHOD FOR DERACEMIZATION OF ENANTIOMER MIXTURES

(75) Inventors: Christian Gruber, Graz (AT); Wolfgang Kroutil, Graz (AT); Constance Voss, Graz (AT); Jan Pfeffer, Essen (DE); Uwe Dingerdissen, Seeheim (DE)

(73) Assignee: EVONIK DEGUSSA GMBH, Essen (DE)

(21) Appl. No.: 12/866,612

(22) PCT Filed: Mar. 26, 2009
(86) PCT No.: PCT/EP2009/053576

§ 371 Date: Dec. 2, 2010

(30) Foreign Application Priority Data
Apr. 1, 2008 (AT) A503/2008

(51) Int. Cl. C12P 41/00

(52) U.S. Cl. 435/280

(57) ABSTRACT

The invention relates to a process for enzymatic deracemization of enantiomer mixtures of secondary alcohols by a combination of oxidation and reduction reactions by means of stereoselective alcohol dehydrogenases and the cofactors thereof, wherein one enantiomer of an optically active secondary alcohol is in a formal sense selectively oxidized to the corresponding ketone, which is subsequently reduced selectively to the optical antipode, while the reduced form of the cofactor is provided for the reduction reaction by means of an additional enzyme, characterized in that two alcohol dehydrogenases with opposite stereoselectivity and different cofactor selectivity and the two corresponding, different cofactors are used for the oxidation and reduction reactions, and the oxidized and reduced cofactors are interconverted in a parallel enzymatic reaction with the additional enzyme, the direction of the deracemization toward one of the two enantiomers being controllable by the selection of the two alcohol dehydrogenases or using the selectivity difference of the additional enzyme for the two cofactors.

### FIELD OF THE INVENTION

The present invention relates to a process for deracemizing enantiomer mixtures using enzyme systems.

### STATE OF THE ART

In the field of stereoisomerism, considerable advances have been achieved in recent times in racemization, i.e. conversion of an optical isomer to its counterpart in order to obtain a racemic mixture, and deracemization, the exact reverse of this procedure. While a racemization in the case of stereolabile compounds, for instance cyanohydrins, hemi(thio)-acetals, α-substituted carbonyl compounds and α-substituted hydantoins, is achievable by simple, gentle acid or base catalysis, stereostable compounds, for example secondary alcohols and chiral amines, are much more difficult to racemize.

The latter has been possible, for example, by means of transition metal complex-catalyzed redox processes in which one enantiomer which is naturally sp³-hybridized at the chiral center is converted via a prochiral sp²-hybridized intermediate to the other. See, for example, the studies by O. Pamies and J. E. Bäckvall, Trends Biotechnol. 22, 130-135 (2004) and Chem. Rev. 103, 3247-3261 (2003); H. Pellissier, Tetrahedron 59, 8291-8327 (2003); M. J. Kim, Y. Ahn and J. Park, Curr. Opin. Biotechnol. 13, 578-587 (2002); V. Zimmermann, M. Beller and U. Kragl, Org. Process Res. Dev. 10, 622-627 (2006); Y. Asano and S. Yamaguchi, J. Am. Chem. Soc. 127, 7696-7697 (2005).

In the field of biosynthesis, which is inherently highly specific, only a few “true” racemases are known, since there is barely any need for racemization in nature—in contrast to industry. For example, a few specific enzymes for catalysis of the racemization of α-hydroxycarboxylic acids (for example mandelic acid derivatives), α-amino acids and hydantoins are known (see, for example, B. Schnell, K. Faber and W. Kroutil, Adv. Synth. Catal. 345, 653-666 (2003)). For the racemization of secondary alcohols and primary amines, virtually no defined enzymes were known for a long time.

The research group of the present inventors disclosed, in Chem. Eur. J. 13, 8271-8276 (2007), a new racemization strategy which was based on a thermodynamic view instead of a kinetic view of the reactions which proceed: in a reaction system which consists of the two enantiomers R and S and a prochiral intermediate P, each of the two optical antipodes is in chemical and thermodynamic equilibrium with the intermediate, i.e. P⇄S and R⇄P. Using several combinations of two alcohol dehydrogenases of opposite enantioselectivity (referred to hereinafter as ADHs for short), which utilize the same cofactor, either NAD or NADP (nicotinamide adenine dinucleotide (phosphate)), it was possible to racemize different optically active secondary alcohols, including acyloins. The alcohol/ketone equilibrium is kept on the side of the alcohol by suitable selection of the amount and of the ratio between oxidized and reduced form of the cofactor, i.e. NAD⁺:NADH and NADP⁺:NADPH; cf. FIG. 1 for explanation.

When the proportion of NAD(P)⁺ was set to a minimum, proceeding from the pure (S)-isomer, the desired racemate was obtained after a few hours of reaction time. The amount of ketone intermediate was lowered to below 10%, and in some cases to below 1%. Comparative experiments with only one highly selective ADH, in contrast, failed for most of the ADHs tested. Only in one case was a yield of 82% ee (enantiomeric excess, i.e. optical yield) achieved after a reaction time of 14 days.

In an only recently published study by the present inventors (C. V. Voss, C. C. Gruber and W. Kroutil, Angew. Chem. Int. Ed. 47, 741-745 (2008)), the deracemization of racemates of secondary alcohols via a prochiral ketone as an intermediate using a tandem system composed of enantioselective bacterial enzymes for alcohol oxidation in the form of Alcaligenes faecalis cells, a stereoselective ADH and NAD as a cofactor is disclosed. The cofactor was effectively “regenerated”, i.e. returned from the oxidized to the reduced form, by allowing an oxidation of glucose to gluconolactone or gluconic acid catalyzed by means of glucose dehydrogenase (hereinafter, GHD for short) as an “additional enzyme” or “auxiliary enzyme” to proceed in parallel; cf. FIG. 2 for explanation.

In initial experiments using lyophilized Alcaligenes faecalis cells, surprisingly, no deracemization but instead racemization of enantiomerically pure alcohols as the starting substrates was found, which was attributed to an increase in the cell permeability as a result of the lyophilization. Using freshly harvested cells with an intact cell membrane, such that oxidation and reduction proceeded separately from one another, it was possible to convert racemates of different secondary alcohols selectively and in yields of >99% ee to the desired enantiomer.

However, this prior art has several disadvantages. Firstly, the Alcaligenes faecalis system, which provides an enzyme mixture for the oxidation, cannot be defined in exact terms, such that there can be considerable variations in the reactions which proceed, and the reproducibility is therefore not all that high.

Secondly, in all existing processes, 1 mol of oxygen for the oxidation and, stoichiometrically, 1 mol of glucose are consumed for the cofactor regeneration per mole of alcohol isomerized, and 1 mol of gluconic acid or gluconolactone is additionally obtained as a by-product.

It was therefore an object of the invention to provide an improved deracemization process which avoids the above disadvantages.

### DESCRIPTION OF THE INVENTION

It has been found that, surprisingly, this object is achieved by an improved process for enzymatic deracemization of enantiomer mixtures of secondary alcohols by a combination of oxidation and reduction reactions by means of stereoselective alcohol dehydrogenases and the cofactors thereof, wherein one enantiomer of an optically active secondary alcohol is in a formal sense selectively oxidized to the corresponding ketone, which is subsequently reduced selectively to the optical antipode, while the reduced form of the cofactor is provided for the reduction reaction by means of an additional enzyme. The process according to the invention is characterized in that two alcohol dehydrogenases with opposite stereoselectivity and different cofactor selectivity and the two corresponding, different cofactors are used for the oxidation and reduction reactions, and the oxidized and reduced cofactors are interconverted in a parallel enzymatic reaction with the additional enzyme, the direction of the deracemization toward one of the two enantiomers being controllable by the selection of the two alcohol dehydrogenases or using the selectivity difference of the additional enzyme for the two cofactors.

By the process according to the invention, it is possible to achieve deracemizations with virtually quantitative optical yield, i.e. >99% ee, without reagents being consumed stoichiometrically in the course of the parallel reactions as soon as the system has attained a stable equilibrium, as will be explained in detail later. Moreover, exactly defined, pure enzymes (the two ADHs and the additional enzyme) are used for catalysis, which results exclusively in reversible reactions in the process and excellent reproducibility. And finally, the process can be performed in simple one-pot reactions, not requiring separations between the individual component reactions in terms of time or space.

The alcohol dehydrogenases used are preferably commercially available or readily obtainable alcohol dehydrogenases, for example bacterial enzymes from strains of Bacillus, Pseudomonas, Corynebacterium, Rhodococcus, Lactobacillus and/or Thermoanaerobium, for example those from strains of Rhodococcus ruber, Lactobacillus kefir or Thermoanaerobium brockii or enzymes from yeast strains, such as Aspergillus, Candida, Pichia or Saccharomyces, since these gave particularly good results in relation to enantiomeric excess and reaction rate. However, a crucial requirement for the selection of suitable ADH pairs is in particular that the two ADHs must have opposite stereoselectivity and different cofactor selectivity. The cofactors arise correspondingly from the particular selection of the ADHs, are generally NAD and NADP, and are preferably used only in catalytic amounts.

FIG. 3 illustrates the reactions in the process of the invention, where HTS stands for “Hydride Transfer System”, which is understood to mean the side reactions for “regeneration” of the cofactors, which are catalyzed by the additional enzyme designated “Aux”, i.e. interconversion of the oxidized and reduced forms. k₁ to k₄ represent formal rate constants of the first-order reactions of the hydride transfer system.

As outlined in FIG. 3 A, the oxidation and reduction reactions of the secondary alcohol enantiomers are catalyzed by the two ADHs of opposite stereoselectivity (not shown in the scheme). When the reaction of the (S)- to give the (R)-enantiomer proceeds, the oxidation of the (S)-isomer of the (S)-selective ADH which has a cofactor preference for NAD eliminates a hydride ion from the alcohol and transfers it to the oxidized form of the cofactor, NAD⁺, which is converted as a result to the reduced form, NADH. Essentially simultaneously, the additional enzyme Aux abstracts this hydride ion from NADH (which gives “Aux-H”) and then transfers it to the second cofactor in the oxidized form, NADP⁺, which provides the reduced form thereof, NADPH. This in turn transfers the hydride, by means of the second, (R)-selective ADH with NADP preference, to the ketone intermediate P, which reduces it to the (R)-enantiomer. In the reverse direction, i.e. in the conversion of the (R)-isomer to the (S)-form, the opposite reactions of course proceed analogously.

If an enzyme/cofactor system in equilibrium is assumed, one and the same hydride ion passes through the reactions explained above and finally arrives back at a now stereoinverted alcohol molecule.

The transfer of the hydride ion by the additional enzyme from one cofactor to the other proceeds in the above-described simple form when the additional enzyme is a nucleotide transhydrogenase. In this case, Aux-H in FIG. 3 B represents a complex of the enzyme with the hydride ion. Since the nucleotide transhydrogenases tested, however, did not give satisfactory results, the inventors found, in their search for alternatives, that, instead of the nucleotide transhydrogenase which directly transfers the hydride, it is also possible for a further dehydrogenase/substrate system to assume the role thereof. In this case, Aux-H represents the reduced form of the substrate corresponding to the additional enzyme.

Useful such additional enzymes in principle include all cofactor-dependent oxidoreductases which do not disrupt the oxidation and reduction reactions of the secondary alcohol to be deracemized. Dehydrogenases, preferably glucose dehydrogenases (GDH), glucose 6-phosphate dehydrogenase (G6PDH) and formate dehydrogenase (FDH), gave very good results and are therefore preferred additional enzymes.

In the first two cases, as a result of the hydride transfer to the substrate, gluconic acid or gluconolactone or the 6-phosphate thereof is reduced to glucose or glucose 6-phosphate (which gives “Aux-H”) and immediately oxidized again. The situation is similar in the third case with CO₂, which is in equilibrium with formate as Aux-H. Although the reaction equilibrium of the oxidation of formate to CO₂ is far to the carbon dioxide side, the reversibility of the reaction in principle was confirmed. Since no (or barely any) additional substrate is consumed in the process according to the invention and therefore only small amounts are required, the formate dehydrogenase/formate/CO₂ system is entirely suitable for the present purposes, as the later examples will show.

As mentioned above, the process according to the invention does not result in stoichiometric consumption of the reagents, as soon as the equilibrium state has been attained. Since this depends on the specific enzyme/substrate combination and the selectivities thereof, a forecast or preadjustment is impossible. Therefore, in practice, this equilibrium is established at the start of the deracemization process. In this phase, which typically lasts a few minutes, a small amount of additional substrate, i.e., glucose or gluconic acid, formate or CO₂, is indeed consumed.

The direction in which the isomerization of the secondary alcohol proceeds depends primarily on stereoselectivity and cofactor selectivity of the two ADHs, but subsequently also on the different selectivity of the additional enzyme for the two cofactors. For the examples shown in the above scheme, which proceeds from a (S)-selective ADH with NAD preference and an (R)-selective ADH with NADP preference, the direction of the deracemization can indeed be preset by the cofactor selectivity of the additional enzyme, which is why this can quite appropriately also be referred to as the “control enzyme”. The cofactor selectivity in the oxidation or reduction mode of the additional enzyme leads either to the effect that NADH and NADP⁺ are converted to NAD⁺ and NADPH (in FIG. 3 B: k₁+k₃>k₂+k₄), or to the effect that NAD⁺ and NADPH are converted to NADH and NADP⁺ (in FIG. 3 B: k₁+k₃<k₂+k₄), which leads in the former case to the formation of the (R)-enantiomer and in the other case to the formation of the (S)-enantiomer. When the control enzyme or substrate thereof is omitted or the control enzyme has no selectivity for one of the two cofactors (which is admittedly extremely improbable), not only no deracemization whatsoever but—proceeding from optically pure alcohols—actually the reverse reaction, i.e. racemization, is observed, as is also evident from FIG. 4: FIGS. 4 A and 4 B show the reaction profiles with 2-octanol as the secondary alcohol for one formate dehydrogenase each, the latter having opposite cofactor selectivity, and FIG. 4 C shows that for a system without FDH.

The direction of the deracemization can, however, also be reversed by a selection of ADH pairs with a reversal of the opposite stereoselectivity or cofactor selectivity. When, for example, in the above scheme—with the same additional enzyme—an (S)-selective ADH with NADP preference and an (R)-selective ADH with NAD preference are used, the racemate selectively forms the optically pure (S)-enantiomer instead of the (R)-enantiomer.

The process according to the invention is typically performed in a solvent selected from the group comprising water, mono- or polyphasic mixtures of water and one or more organic solvents, and ionic liquids, though preference is given to using a conventional aqueous buffer system for reasons of cost and stability.

An aqueous buffer system is understood to mean an aqueous solvent which contains substances, for example salts, which make the solvent insensitive to pH changes. Known aqueous buffer systems are, for example, the carbonic acid/bicarbonate system, the carbonic acid-silicate buffer, the acetic acid/acetate buffer, the phosphate buffer, the Michaelis veronal/acetate buffer, the ammonia buffer, HEPES (4-(2-hydroxyethyl)-1-piperazineethanesulfonic acid) and MES (2-(N-morpholino)ethanesulfonic acid.

### BRIEF DESCRIPTION OF THE FIGURES

FIG. 1 shows: principle of the enzyme-catalyzed racemization by means of two specific ADHs under via the prochiral ketone

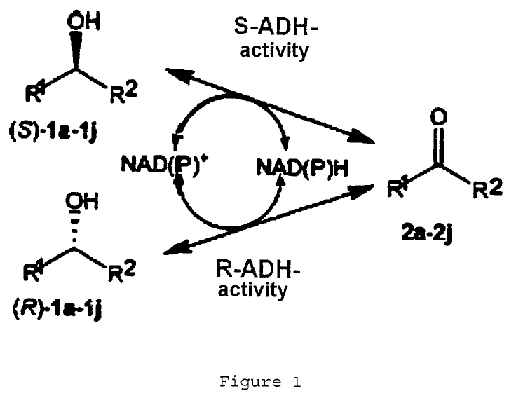

FIG. 2 shows: optical resolution with defined strain background and subsequent cofactor regeneration

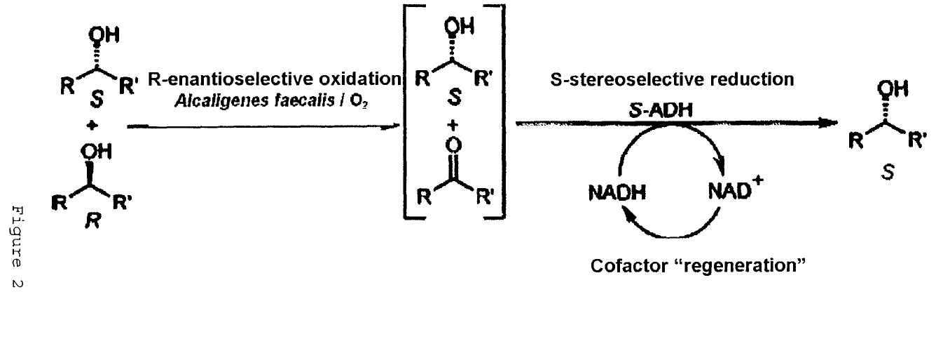

FIG. 3 shows: A: principle of the inventive enzyme-catalyzed deracemization by means of two specific ADHs and auxiliary enzyme (hydride transfer system: HTS) for cofactor regeneration, B: principle of the hydride transfer system

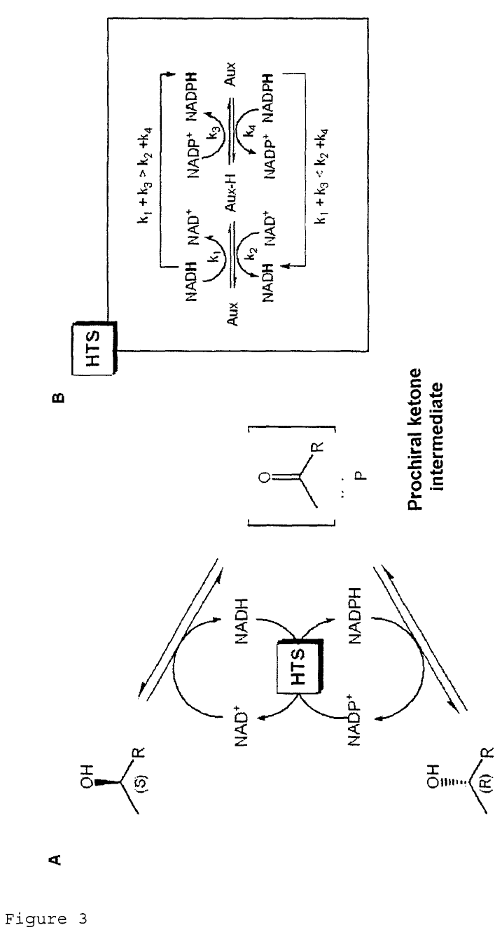

FIG. 4 shows: shift of the racemic mixture of 1-phenylethanol: A and B: with formate dehydrogenase (FDH), C: without formate dehydrogenase (FDH)

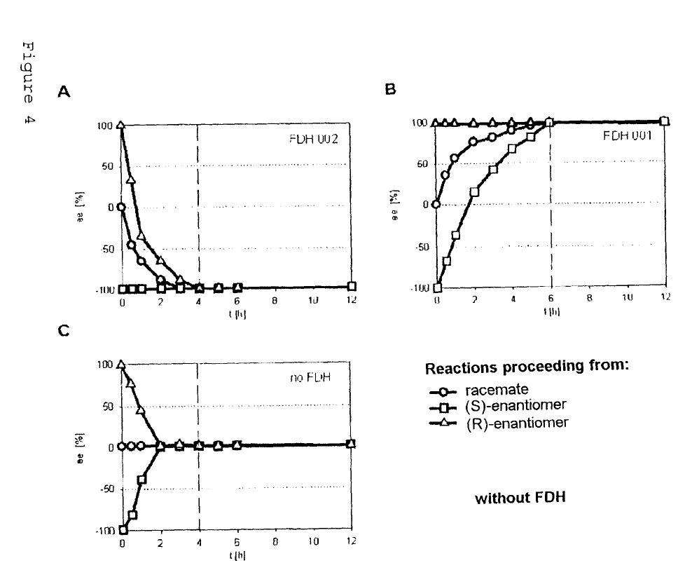

FIG. 5 shows: shift in the racemic mixture by means of NAD- or NADP-specific formate dehydrogenase (FDH)

FIG. 6 shows: influence of the variation of different reaction parameters on the reaction equilibrium

FIG. 7 shows: influence of different concentrations of the additional glucose substrate on the establishment of the racemic equilibrium, A: plot against time for different glucose concentrations, B: correlation of ee [%] with the particular glucose concentrations after 6 hours of reaction time.

### EXAMPLES

The invention is now described in detail with reference to representative, nonlimiting working examples.

### Materials, Sources and Process

### Enzymes

ADH-A: Alcohol dehydrogenase from Rhodococcus ruber (commercially available from BioCatalytics Inc., now Codexis, Pasadena, USA).
LK-ADH: Alcohol dehydrogenase from Lactobacillus kefir (commercially available from Sigma-Aldrich, Vienna, #05643, 0.4 IE/mg).
RE-ADH: Alcohol dehydrogenase from Rhodococcus erythropolis (commercially available from Sigma-Aldrich, #68482, 20 IE/ml).
LB-ADH: Alcohol dehydrogenase 002 (commercially available from Jülich Chiral Solutions, now Codexis, #05.11).
ADH-T: Alcohol dehydrogenase 005 (commercially available from Jülich Chiral Solutions, now Codexis, #26.10).
ADH-PR2: Alcohol dehydrogenase 007 (commercially available from Jülich Chiral Solutions, now Codexis, #42.10).
TB-ADH: Alcohol dehydrogenase from Thermoanaerobium brockii (commercially available from Sigma-Aldrich, #A9287, 30-90 IE/mg).
G6PDH: Glucose 6-phosphate dehydrogenase from baker's yeast (commercially available from Sigma-Aldrich, #49271, 240 IE/mg).
GLY-DH: Glycerol dehydrogenase from Geotrichum candidum (commercially available from Sigma-Aldrich, #49860, 30 IE/mg).
LDH-SC: D-Lactate dehydrogenase from Staphylococci (commercially available from Sigma-Aldrich, #17847, 120 IE/mg).
LDH-LS: D-Lactate dehydrogenase from Lactobacillus sp. (commercially available from Sigma-Aldrich, #59023, 400 IE/mg).
LDH-RM: L-Lactate dehydrogenase from rabbit muscle (commercially available from Sigma-Aldrich, #61311, 500 IE/mg).
FDH1: NADP-specific formate dehydrogenase 001 (commercially available from Jülich Chiral Solutions, now Codexis, Pasadena, USA, #25.10, 47 IE/ml).
FDH2: NAD-specific formate dehydrogenase 002 (commercially available from Jülich Chiral Solutions, now Codexis, #24.11, 200 IE/ml).
FDH3: NAD-specific formate dehydrogenase 001 (commercially available from Jülich Chiral Solutions, now Codexis, #09.11, 200 IE/ml).
FDH4: Formate dehydrogenase from yeast (commercially available from Boehringer Mannheim GmbH, #204226, 0.5 IE/mg).
FDH5: Formate dehydrogenase from Candida boidinii (gift from Martina Pohl, University of Düsseldorf, Germany).
GDH-BM: D-Glucose dehydrogenase 001 (commercially available from Jülich Chiral Solutions, now Codexis, #22.10, 30 IE/mg).
GDH-BS: D-Glucose dehydrogenase 002 (commercially available from Jülich Chiral Solutions, now Codexis, #29.10, 500 IE/ml).

### Properties of the Enzymes

TABLE 1
Alcohol dehydrogenases

| No. | Enzyme | Cofactor selectivity | Stereoselectivity |
|---|---|---|---|
| 1 | ADH-A | NAD | (S) |
| 2 | LK-ADH | NADP | (R) |
| 3 | RE-ADH | NAD | (S) |
| 4 | LB-ADH | NADP | (R) |
| 5 | ADH-T | NADP | (S) |
| 6 | ADH-PR2 | NAD | (R) |
| 7 | TB-ADH | NAD | (R) |

TABLE 2
Additional enzymes

| | | | Reduced additional |
|---|---|---|---|
| No. | Enzyme | Cofactor selectivity^{[a]} | substrate |
| 1 | FDH1 | NADP | formate |
| 2 | FDH2 | NAD | formate |
| 3 | FDH3 | NAD | formate |
| 4 | FDH4 | unknown^{[b]} | formate |
| 5 | FDH5 | unknown^{[b]} | formate |
| 6 | GDH-BS | NAD and NADP | α-D-glucose |
| 7 | GDH-BM | NAD and NADP | α-D-glucose |
| 8 | G6PDH | NADP | α-D-glucose |
| | | | 6-phosphate |
| 9 | GlyDH | NAD | glycerol |
| 10 | LDH-SC | unknown^{[b]} | lactate |
| 11 | LDH-LS | unknown^{[b]} | lactate |
| 12 | LDH-RM | unknown^{[b]} | lactate |

^{[a]}Data either from the literature or from the manufacturer.
^{[b]}No data found

### Chemicals

rac-2-Octanol (#04504, MW 130.23 g/mol), (R)-2-octanol (#74864, MW 130.23 g/mol), (S)-2-octanol (#74863, MW 130.23 g/mol), 2-octanone (#53220, MW 128.21 g/mol), ammonium formate (#09739, 63.06 g/mol), sodium formate (#3996-15-4, 69.02 g/mol) and formic acid as the potassium salt (#57444-81-2, MW 85.13 g/mol) were purchased from Sigma-Aldrich, Vienna.

Chemicals for extraction and workup:

Ethyl acetate (#441977) for extraction was purchased from Brenntag CEE GmbH, Ort, and used in freshly distilled form. DMAP (#29224, MW 122.17 g/mol) and acetic anhydride (#45830, MW 102.09 g/mol) for acetylation were purchased from Sigma-Aldrich, Vienna.

### General Procedure

Model process for shifting the optical composition: The activity of the commercial enzymes is generally reported in international units (IE). However, all of these units report the activity of the particular enzyme for a different substrate than used herein. The activity of the enzymes used in the reduction of 2-octanone with a suitable “regeneration” system was therefore determined (generally an FDH with ammonium formate, 5 eq.). For all experiments, about 1 IE_{2-octanol} of the ADHs was used.

System 1: ADH-A, LK-ADH, NADP-specific FDH (2 IE), ammonium formate (3 eq. of the substrate concentration), NAD⁺ and NADP⁺ (3 mol % of the substrate) were suspended in TRIS-HCl (50 mM, pH 7.5, total volume 0.5 ml). The reaction was started by adding racemic 2-octanol (0.5 μl, 8 mmol/ml, ee <3%). After shaking (130 rpm) at 30° C. for 3 h, the mixture was extracted with EtOAc (500 μl) and centrifuged in order to bring about phase separation.
System 2: As system 1 apart from the use of an NAD-specific FDH (2 IE).
System 3: As system 1 apart from the use of GDH-BS (2 IE) and α-D-glucose (1 eq., 8 mmol/l).
System 4: As system 1 apart from the use of ADH-T and ADH-PR2.
System 5: As system 1 apart from the use of ADH-T, ADH-PR2 and an NAD-specific FDH.
System 6: As system 1 apart from the use of RE-ADH and Thermoanaerobium brokii ADH.

### Analysis Methods

### Chiral GC-FID Analysis:

The alcohols were acetylated by adding acetic anhydride (100 ml) and DMAP (0.5 mg) at 30° C. within 2 h. After the workup, the products were analyzed by means of GC-FID and GC-MSD with a chiral stationary phase.

Chiral GC-FID analyses were effected on a Varian 3900 gas chromatograph with an FID detector using a Chrompack Chirasil DEX CB column (Varian, 25 m×0.32 mm×0.25 mm, 1.0 bar H₂), detector temperature 250° C., split ratio 90:1.

### Chiral GC-MSD Analysis:

Chiral GC-MSD analyses were effected on an Agilent 7890A GC system with a mass-selective Agilent 5975C detector and an FID using a Chrompack Chirasil DEX CB column (Varian 25 m×0.32 mm×0.25 mm, 1.0 bar H₂), detector temperature 250° C., split ratio 90:1.

### Chiral HPLC Analysis:

HPLC analyses were effected on a Shimadzu HPLC system with a DGU-20A5 degasser, LC-20AD liquid chromatograph, SIL-20AC autosampler, CBM-20A communication bus module, SPD-M20A diode array detector and CTO-20AC column oven using a Chiralpak AD column (Daicel, 0.46×25 cm) with n-heptane/isopropanol=90:10, 0.5 ml/min, 18° C.

### Examples 1 to 13

### Comparative Examples 1 to 4

Deracemizations were carried out using different ADH/additional enzyme combinations with 2-octanol as the secondary alcohol under the following conditions: substrate concentration 8 mmol/l, reaction time 3-12 h, 30° C. in TRIS-HCl (pH 7.5, 50 mM) or phosphate buffer (pH 7.5, 50 mM), shaking at 130 rpm. About 1 IE for each of the ADHs (for 2-octanol as the substrate); NAD⁺ and NADP⁺ in catalytic amounts (approx. 3 mol %). Additional enzymes: 2 IE (for the natural substrate thereof, as reported by the manufacturer). Additional substrate (formate, glucose, glucose 6-phosphate, lactate and glycerol): 16 mmol/l. The compositions and results are compiled in table 3 below.

TABLE 3
Deracemization of rac-2-octanol

| | | Enzymes | | | |
|---|---|---|---|---|---|
| Example/ | | Alcohol | | Product | |
| comp. | Sys- | dehydrogenases, | Additional | ee | Alcohol |
| example | tem | ADHs | enzyme | [%] | [%] |
| E1 | 1 | ADH-A + | NADP-specific | >99 | >99% |
| | | LK-ADH | FDH 001 | (R) | |
| E2 | | | NAD-specific | >99 | >99% |
| | | | FDH 002 | (S) | |
| E3 | | | NAD-specific | >99 | >99% |
| | | | FDH 001 | (S) | |
| E4 | | | FDH4 | 61 | >99% |
| | | | | (S) | |
| E5 | | | GDH-BS | >99 | >99% |
| | | | | (R) | |
| E6 | | | GDH-BM | 41 | >99% |
| | | | | (R) | |
| E7 | | | G6P-DH | >99 | >99% |
| | | | | (R) | |
| C1 | | | LDH-SC | rac | >99% |
| C2 | | | LDH-LS | rac | 96% |
| C3 | | | LDH-RM | rac | >99% |
| C4 | | | GlyDH | rac | 97% |
| E8 | 2 | ADH-T + | NADP-specific | >99 | >99% |
| | | ADH-PR2 | FDH 001 | (S) | |
| E9 | | | NAD-specific | >99 | >99% |
| | | | FDH 002 | (R) | |
| E10 | 3 | RE-ADH + | NADP-specific | 94 | >99% |
| | | LB-ADH | FDH 001 | (R) | |
| E11 | | | NAD-specific | 34 | >99% |
| | | | FDH 002 | (S) | |
| E12 | 4 | Thermoanaerobium | NADP-specific | 89 | >99% |
| | | brokii ADH + | FDH 001 | (S) | |
| | | ADH-PR2 | | | |
| E13 | | | NAD-specific | 96 | >99% |
| | | | FDH 002 | (R) | |

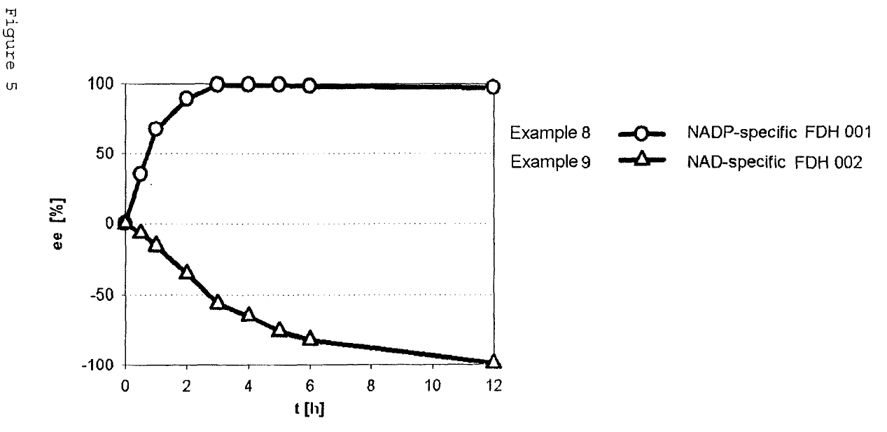

It is evident that the enzyme combinations tested in examples 1 to 13 of the present invention afforded one of the enantiomers from 2-octanol racemates in predominantly good, in some cases virtually quantitative enantiomeric excess and almost always in quantitative yield. In the case of use of the same ADH pair, reversal of the cofactor specificity of the additional enzyme allowed the direction of the deracemization to be controlled: cf. examples 1/2+3, 8/9, 10/11, 12/13. The result of examples 8 and 9 is also shown in graphic form in FIG. 5.

In the case of use of lactate dehydrogenase or glycerol dehydrogenase in comparative examples 1 to 4, in contrast, there was no deracemization whatsoever.

### Examples 14 to 22

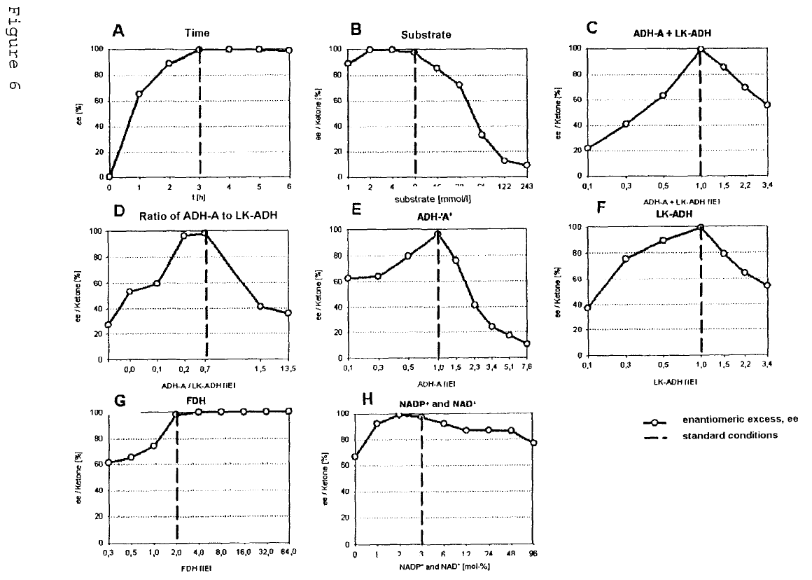

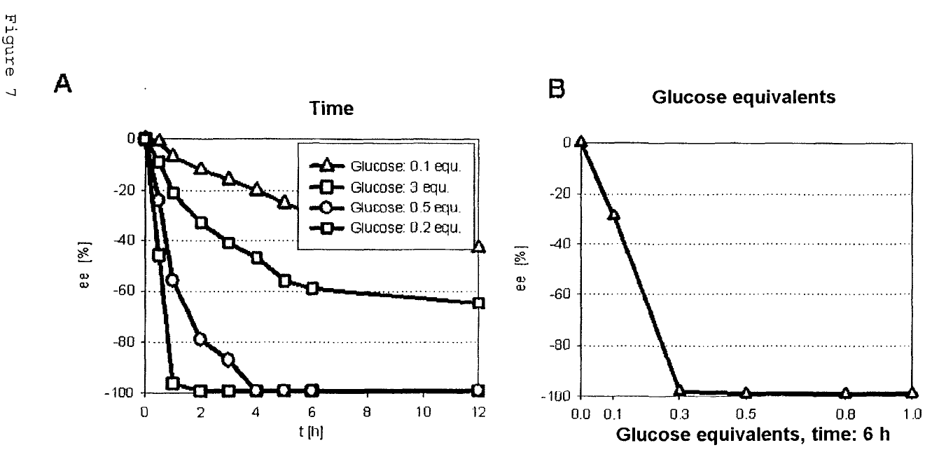

In these examples, different reaction parameters were varied using the enzyme system from example 1 in order to study the effect thereof on the course of the reaction. The results of examples 14 to 21 are shown in graphic form in FIG. 6 A-H, and those of example 22 in FIG. 7 A-B.

### Example 14

The reaction time was varied here between 1 and 6 h, and it was found that quantitative conversion had already been attained after 3 h. The further examples of this group were therefore carried out for 3 h (FIG. 6 A).

### Example 15

The alcohol concentration was varied between 1 and 243 mmol/l, and 2 to 8 mmol/l gave the best results with the given reaction time of 6 h. At higher concentrations, either a longer reaction time or a greater amount of enzyme is needed in order to achieve full conversion (FIG. 6 B).

### Example 16

The total amount of the two ADHs was varied between 0.1 and 3.4 IE, and 1 IE was found to be the optimal activity amount (FIG. 6 C).

### Example 17

The activity ratio (in IE) of the two ADHs to one another was varied between 0.01 and 13.5, and it was found that a ratio between about 0.2 and about 0.7 was the most effective, although a value for a 1:1 ratio was absent (FIG. 6 D).

### Examples 18 and 19

The activity of one of the two ADHs in each case was varied between 0.1 and 7.6 or 3.4 IE, with an activity of the second ADH of 1 IE, and it was found that 1 IE also constitutes the optimal activity amount for the second enzyme, and 1:1 is therefore the optimal activity ratio of the two ADHs (FIG. 6 E, 6 F).

### Example 20

The amount of FDH was varied between 0.3 and 64.0 IE, and it was found that quantitative conversion was already achieved from an amount of 2 IE (FIG. 6 G).

### Example 21

The combined concentration of the cofactors NAD and NADP was varied between 0 and 96 mol %, and a concentration of about 2 to 3 mol % was found to be the most effective (FIG. 6 H).

### Example 22

Example 5 was repeated, except that the concentration of the additional substrate, i.e. glucose, was varied between 0.1 and 3 equivalents of the alcohol concentration over a reaction time between 0.5 and 12 h, as shown in FIG. 7 A. The enantiomeric excess ee after 6 h with variation of the glucose equivalents between 0.1 and 1 is shown in FIG. 7 B. >99% ee was already achieved from 0.3 equivalent, which shows that a distinctly substoichiometric proportion of additional substrate is also sufficient.

### Examples 23 to 32

In these examples, using the enzyme system from example 1, deracemization of racemates of 10 other secondary alcohols was attempted. The selection of the alcohols was made taking account of the substrate spectra described in the literature for the two ADHs involved. In principle, it should be possible in this way to deracemize, by the process according to the invention, all substrates present in the substrate spectrum of both ADHs. The structures of the secondary alcohols used in these examples are listed in table 4 below.

TABLE 4
Substrates of examples 1 and 23 to 32

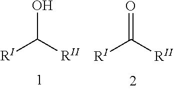

| Example | R^{I} | R^{II} | Name |
|---|---|---|---|
| 1 | CH₃ | C₆H₁₃ | 2-Octanol |
| 23 | CH₃ | C₇H₁₅ | 2-Nonanol |
| 24 | CH₃ | C₈H₁₇ | 2-Decanol |
| 25 | CH₃ | 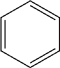 | 1-Phenyl-1-ethanol |
| 26 | CH₃ | 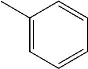 | 1-Phenyl-2-propanol |
| 27 | CH₃ | 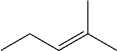 | Sulcatol |
| 28 | C₂H₅ | C₅H₁₁ | 3-Octanol |
| 29 | C₂H₅ | C₆H₁₃ | 3-Nonanol |
| 30 | C₂H₅ | C₇H₁₅ | 3-Decanol |
| 31 | 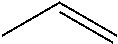 | C₆H₁₃ | 1-Octen-3-ol |
| 32 | -”- | C₅H₁₁ | 1-Hepten-3-ol |

The results of the racemizations are compiled in table 5 below.

TABLE 5
Results of the deracemization of racemates of secondary alcohols

| | | Time | Alcohol | Enan- | ee |
|---|---|---|---|---|---|
| Substrates^{a)} | Example | [h] | [%] | tiomer | [%] |
| rac-2-Octanol | 1 | 3 | 99 | (R) | >99 |
| rac-2-Nonanol | 23 | 3 | 97 | (R) | >99 |
| rac-2-Decanol | 24 | 3 | 99 | (R) | >99 |
| rac-1-Phenylethanol | 25 | 2 | >99 | (R) | >99 |
| rac-1-Phenyl-2-propanol | 26 | 2 | 98 | (R) | 80.5 |
| rac-Sulcatol | 27 | 3 | 95 | (R) | >99 |
| rac-3-Octanol | 28 | 4 | 98 | (R) | >99 |
| rac-3-Nonanol | 29 | 4 | 97 | (R) | >99 |
| rac-3-Decanol | 30 | 4 | >99 | (R) | 98 |
| rac-1-Octen-3-ol | 31 | 3 | 99 | (S)^{b)} | 95 |
| rac-1-Hepten-3-ol | 32 | 3 | 93 | (S)^{b)} | 96 |

^{a)}ee of the racemic substrates <3%.
^{b)}change in the Cahn-Ingold-Prelog priority

It is clearly evident from the table that all racemates tested can be deracemized virtually quantitatively in a short time with excellent selectivity by the process according to the invention, and the presence of further functionalities did not detract from the efficacy of the process according to the invention.

The present invention thus constitutes a valuable enrichment to the field of stereoisomerization, and there is therefore no doubt about the industrial applicability of the invention.

1. A process for enzymatic deracemization of mixtures of enantiomers of secondary alcohols by a combination of oxidation and reduction reactions by stereoselective alcohol dehydrogenases and cofactors thereof, wherein one enantiomer of an optically active secondary alcohol is in a formal sense selectively oxidized to a corresponding ketone, which is subsequently reduced selectively to an optical antipode of the one enantiomer of the optically active secondary alcohol, while a reduced form of a cofactor is provided for a reduction reaction by an additional enzyme,
wherein two alcohol dehydrogenases with opposite stereoselectivity and different cofactor selectivity and two corresponding, different cofactors are employed for the oxidation and reduction reactions, and oxidized and reduced cofactors are interconverted in a parallel enzymatic reaction with the additional enzyme, a direction of the deracemization toward one of the two enantiomers being controlled by selection of the two alcohol dehydrogenases or utilizing a selectivity difference of the additional enzyme for the two cofactors.

2. A process according to claim 1, wherein the alcohol dehydrogenases employed are bacterial alcohol dehydrogenases.

3. A process according to claim 1, wherein the alcohol dehydrogenases employed are alcohol dehydrogenases from Bacillus, Pseudomonas, Corynebacterium, Rhodococcus, Lactobacillus, or Thermoanaerobium.

4. A process according to claim 1, wherein the alcohol dehydrogenases employed are enzymes from yeast strains.

5. A process according to claim 4, wherein the alcohol dehydrogenases employed are alcohol dehydrogenases from Aspergillus, Candida, Pichia, or Saccharomyces.

6. A process according to claim 1, wherein the two alcohol dehydrogenases are employed in an activity ratio of 1:1.

7. A process according to claim 1, wherein the two alcohol dehydrogenases are employed in a total amount of 1 IE.

8. A process according to claim 1, wherein a racemate of the secondary alcohol is employed in a concentration of at least 2 mmol/L.

9. A process according to claim 1, wherein the additional enzyme employed is a glucose dehydrogenase, glucose 6 phosphate dehydrogenase, formate dehydrogenase, or nucleotide transhydrogenase.

10. A process according to claim 1, wherein the additional enzyme is employed in an amount of 2 IE.

11. A process according to claim 1, wherein a substrate of the additional enzyme is employed in an amount of at least 0.3 mol per mole of secondary alcohol.

12. A process according to claim 1, wherein the cofactors are employed in catalytic amounts.

13. A process according to claim 9, wherein the cofactors are employed in an amount of 2 to 3 mol %, based on the secondary alcohol.

14. A process according to claim 1, wherein a solvent selected from the group consisting of water, a mono-phasic mixture of water and at least one organic solvent, a biphasic mixture of water and at least one organic solvent, a polyphasic mixture of water and at least one organic solvent, and at least one ionic liquid is employed.

15. A process according to claim 14, wherein the solvent employed is an aqueous buffer system.

16. A process according to claim 2, wherein the alcohol dehydrogenases employed are alcohol dehydrogenases from Bacillus, Pseudomonas, Corynebacterium, Rhodococcus, Lactobacillus, or Thermoanaerobium.

17. A process according to claim 2, wherein the two alcohol dehydrogenases are employed in an activity ratio of 1:1.

18. A process according to claim 3, wherein the two alcohol dehydrogenases are employed in an activity ratio of 1:1.

19. A process according to claim 4, wherein the two alcohol dehydrogenases are employed in an activity ratio of 1:1.

20. A process according to claim 5, wherein the two alcohol dehydrogenases are employed in an activity ratio of 1:1.
